(19) United States
(12) Patent Application Publication
(10) Pub. No.: US 2004/0018581 A1
(43) Pub. Date: Jan. 29, 2004

(54) Method for discovering substances for inhibiting enzymes

(75) Inventors: Timothy Geary, Kalamazoo, MI (US); Margaret Favreau, Richland, MI (US)

Correspondence Address: Patrick G. Gattari, McDonnell Boehnen Hulbert & Berghoff, 32nd Floor, 300 S. Wacker Drive, Chicago, IL, 60606, US

(73) Assignee: Pharmacia and UpJohn Company

(21) Appl. No.: 10/202,348

(22) Filed: Jul. 24, 2002

(51) Int. Cl. C12Q 1/18

(52) U.S. Cl. 435/32

(57) ABSTRACT

This invention describes novel methods for the identification of compounds useful as inhibitors of enzymes. The methods involve obtaining a target gene-complemented microorganism which is dependent on the expression of the target gene for survival in test conditions. By measuring the viability of the complemented microorganism after exposure to a compound, compounds that inhibit growth in test conditions are identified. These compounds that inhibit growth are further tested in conditions where the expression of the target gene is not required for growth in order to identify compounds that specifically inhibit the target gene but do not effect the viability of the host.

### FIELD OF THE INVENTION

The invention relates to a method for discovering substances which inhibit enzymes. More specifically, the invention relates to a method for screening compounds which inhibit the activity of enzymes in the metabolic pathways of parasitic organisms.

### BACKGROUND OF THE INVENTION

The following description refers to a number of references by author and date. Complete citations to the references may be found in the section entitled “References” immediately preceding the claims.

New methods for discovering compounds useful as novel antiparasitic drugs are needed. Of particular and urgent need are broad-spectrum drugs for veterinary diseases caused by parasitic protozoans in the phylum Apicomplexa, specifically, species in the genus Eimeria. These parasites are the causative agents of coccidiosis infecting cattle, sheep and poultry. In the poultry industry, coccidiosis is a major problem and it is currently controlled to some extent by chemoprophylaxis, although the development of resistance has been reported for all classes of anticoccidial drugs (see Chapman, 1997 for review). Recombinant vaccines for the prevention or control of poultry coccidiosis have long been sought but have not yet been widely introduced to the market.

An alternative strategy for reduction of economic losses due to infections with Eimeria is to reduce contamination of the poultry house with viable and infectious oocysts (Wallach, 1997). Treatment of housed poultry with a compound that selectively reduces oocyst production and infectivity will reduce severity and spread of coccidial infections in the next group of birds reared in the house. This mode of treatment may also shorten the interval needed between introduction of successive flocks in a given house.

Potential targets for drugs that inhibit infectious oocyst development have been identified in the biochemical pathways of the various stages of the Eimeria life cycle (Wang, 1997; Coombs, 1999). A unique biochemical feature of this life cycle is the dependence for viability on endogenous energy sources when unsporulated oocysts are shed into the environment. Mannitol, which is present in high amounts in unsporulated oocysts and is consumed during sporulation (Schmatz, 1997; Alloco et al., 1999), serves as an energy source. The mannitol cycle has been described in plants, bacteria, fungi and in protozoan, particularly, the phylum Apicomplexa. Vertebrates and other metazoan animals neither synthesize nor metabolize mannitol.

One of the enzymes of the mannitol pathway, mannitol-1-phosphate dehydrogenase (M1PDH), is present in both unsporulated and sporulated oocysts of Eimeria tenella as well as in the sexual stages of its life cycle. Mannitol-1-phosphate dehydrogenase catalyzes the rate-limiting step in mannitol biosynthesis (Schmatz, 1997; Alloco et al., 1999). Interruption of this step results in loss of parasite viability by preventing the accumulation of mannitol required for the development of infectious oocysts. This principle has been demonstrated through experiments with the known anticoccidial nitrophenide which acts via the inhibition of M1PDH (Schmatz, 1997; Alloco et al., 1999 and 2001).

The Eimeria tenella cDNA encoding M1PDH has been reported (Liberator et al., 1997). Functional expression of this cDNA has been difficult to achieve since the coccidial enzyme forms stable complexes with members of the 14-3-3 protein family when expressed in heterologous systems such as E. coli or in the parasite itself (Liberator et al., 1997; Myers et al., 1997; Schmatz, 1997). This makes high-throughput screening for inhibitors of recombinant M1PDH a problem in standard purified-enzyme protocols. Thus, an alternative screening strategy is needed to circumvent this problem.

One possible solution is to use a complemented microorganism that depends on the expression of a parasite enzyme for survival. Particularly useful parasitic enzymes are those that form part of metabolic pathways well known in the art. One kind of metabolic pathway is that which uses a carbon source to provide energy in a usable form, such as ATP, and to synthesize compounds that are essential to maintain organismic homeostasis. This type of metabolic pathway is characterized by having a number of enzymes working in sequence. Metabolic pathways of this type are common to many different organisms and usually share homologous enzymes performing related functions. It is possible to rescue a mutant microorganism which has a defect in an enzyme by using a homologous enzyme derived from any other organism by a process called complementation. One method by which complementation can be achieved is by providing the mutant microorganism with a plasmid containing a homologous gene coding for the homologous enzyme. Expression of the homologous enzyme enables the mutant microorganism to catalyze the reactions of the endogenous enzyme and be able to survive in normal conditions.

Another form of complementation that leads to rescue of a mutant microorganism is to use a heterologous enzyme that produces an end product that can substitute for the missing endogenous end product. In this case, the end product produced by the heterologous enzyme resembles, structurally or functionally, the end product produced by the endogenous enzyme. This resemblance can be due to similar structural features that enables the heterologous end product to substitute for the endogenous end product or because the heterologous end product can provide the same cellular function as the endogenous end product.

Parasitic organisms are opportunistic in the sense that they exploit the environment of the host to survive. In order to selectively kill such parasites, chemical agents must be able to distinguish parasite from host. One way to achieve this is to target the metabolic pathways that the parasite needs to survive but which are absent from the host or at least are non-essential for host survival.

It has been shown that the homologous m1pdh gene from E. coli, mt1D, is able to complement a strain of Saccharomyces cerevisiae that has a deficient gpd1 gene. The gpd1 gene codes for glycerol-3-phosphate dehydrogenase (Chaturvedi et al., 1997) which catalyzes the first reaction in the biosynthesis of glycerol, conversion of dihydroxyacetone phosphate to glycerol-3-phosphate. Glycerol is required for survival of yeast in conditions of osmotic stress. Functional expression of the E. coli gene encoding M1PDH in the gpd1 deficient strain of yeast restored osmotolerance (Chaturvedi et al., 1997).

Thus, a recombinant microorganism that can be made dependent for survival on the function of a parasite enzyme provides a powerful tool for screening for antiparasitic drugs. Obtaining a deficient yeast strain that depends for survival on the functional expression of Eimeria tenella m1pdh gene could be used to screen for compounds that possess inhibitory properties towards Eimeria tenella M1PDH. Such a strain could be useful for high-throughput screening protocols for identifying novel M1PDH inhibitors.

### SUMMARY OF THE INVENTION

One aspect of the invention is directed to a method for identifying a compound having antiparasitic activity. The method includes exposing to the compound a parasite target gene-complemented microorganism growing in a selection medium that inhibits the viability of non-complemented microorganisms. The viability of the gene-complemented microorganism in the selection medium after exposure to the compound is compared to the viability of the microorganism in the selection medium lacking the compound. Compounds that decrease viability of the microorganism, thus having potential antiparasitic activity, are identified. The method further includes comparing viability of the target gene-complemented microorganism after exposure to the compound in the absence of the selection medium to viability of the microorganism in the absence of both the selection medium and the compound.

In one embodiment of the invention, the target gene expresses an enzyme in the mannitol pathway of a parasite. The target gene may be a mannitol-1-phosphate dehydrogenase (m1pdh) gene from a parasite in the phylum Apicomplexa.

Another embodiment of the invention provides for a method for identifying a compound useful as an antiparasitic drug. The method includes determining whether the compound decreases viability of a target gene-complemented microorganism by comparing the viability of a target gene-complemented microorganism growing in a selection medium after exposure to a compound to the viability of the target gene-complemented microorganism growing in the selection medium in the absence of the compound. Compounds useful as antiparasitic drugs are identified by comparing the viability of the target gene-complemented microorganism after exposure to the compound in the absence of the selection medium to viability of the microoganism in the absence of both the selection medium and the compound.

Yet another embodiment of the invention is a method of screening for a compound or identifying a compound that inhibits an essential parasite gene product required for parasite viability. The method includes rendering a microorganism incapable of growing under test conditions. A target gene-complemented microorganism is produced by complementing the microorganism with parasite gene encoding a parasite gene product that enables the microbial strain to grow in test conditions. The complemented microorganism is then exposed to a compound to be tested for parasite gene product inhibitory properties and comparing the viability of the target gene-complemented microorganism exposed to the compound to the viability of the target gene-complemented microorganism in the absence of the compound. Compounds that inhibit growth of the target gene-complemented microorganism are identified as compounds that inhibit an essential parasite gene product. In a preferred embodiment, the method further includes comparing viability of the target gene-complemented microorganism after exposure to the compound in the absence of the selection medium to viability of the target gene-complemented microorganism in the absence of both the selection medium and the compound.

In one aspect of the invention, the microorganism is unable to produce glycerol in response to osmotic stress. In a further aspect, the microorganism is Saccharomyces cerivisiae mutated in the gene encoding glycerol-3-phosphate dehydrogenase.

Another embodiment of the invention is directed to a polypeptide sequence of mannitol-1-phosphate dehydrogenase from E. tenella. In yet another embodiment, the invention is directed to a polynucleotide sequence encoding, due to the degeneracy of the genetic code, a polypeptide sequence of a mannitol-1-phosphate dehydrogenase from E. tenella. In a further embodiment, the invention is directed to a gene-complemented microorganism which has been transformed with the polynucleotide sequence of a mannitol-1-phosphate dehydrogenase from E. tenella.

### BRIEF DESCRIPTION OF THE FIGURES

FIG. 1 shows the difference in growth of wild-type strain W3031A (left side of the plates) and the Δgpd1 mutant strain (right side of the plate) on YEPD supplemented with increasing concentrations of NaCl.

FIG. 2 shows the growth of wild-type strain W3031A (quadrant B), Δgpd1 mutant strain of W3031A (quadrant A), and transformants Δgpd1-pYES2/m1pdh (quadrant C) and Δgpd1-pYES2/mt1D (quadrant D) in increasing concentration of NaCl after growing in YMM containing raffinose/galactose for 48 hours.

FIG. 3 shows the growth rate of wild-type strain W3031A and transformants Δgpd1-pYES2/m1pdh and Δgpd1-pYES2/mt1D in YEPD medium with and without 1.5 M NaCl. Cells are grown for 24 hours (graybars), 48 hours (black bars) or 72 hours (white bars) in YMM before transferring to YEPD with or without 1.5 M NaCl.

FIG. 4 shows the salt-dependent inhibitory effect of nitrophenide on the growth of yeast transformant Δgpd1-pYES2/m1pdh in YEPD with and without 1.5 M NaCl. Cells are first grown in YMM medium for 48 or 72 hours before plating in YEPD with or without 1.5 M NaCl.

FIG. 5 shows the HPAEC elution profile of mannitol monitored by pulsed amperometric detection. Aqueous monosaccharide extracts of wild-type strain W3031A (panel A), and of transformants Δgpd1-pYES2/mt1D (panel B) and Δgpd1-pYES2/m1pdh (panel C) are injected into CarboPac PA1 analytical column and isocratically eluted with 16 mM NaOH. The retention time of mannitol at approximately 2.7 minutes is determined by using reference monosaccharides and data analysis with Dionex PeakNet 6.0 software. The elutions of trehalose and glucose are also identified at approximate retention times of 3.1 and 13 minutes, respectively.

FIG. 6 shows the amino acid sequence of the M1PDH from Eimeria tenella of the present invention (SEQ ID NO: 6) compared to M1PDH isolated from unsporulated oocyst described by Liberator et al., 1997 in GenBank as protein ID AAD02688 (SEQ ID NO: 8).

FIGS. 7A-D show the polynucleotide sequence of the gene encoding the amino acid sequence of E. tenella mannitol-1-phosphate dehydrogenase of the present invention [SEQ ID NO: 5] compared to the sequence described by Liberator et al, 1997, GenBank Accession No. AF055716 [SEQ ID NO: 7].

### DETAILED DESCRIPTION OF THE INVENTION

The method of the invention is directed to a facile tool to identify candidate anti-parasitic compounds without the need to obtain large quantities of parasites. The screening methods can be performed on customized culture plates avoiding the housing and maintenance of large numbers of test hosts. The method includes a high-throughput screen for specific inhibitors of enzymes of parasite metabolic pathways. Such enzymes are usually essential components of metabolic pathways involved in the survival of the parasite. For example, the enzymes may aid in the utilization of a carbon source for energy or for the synthesis of structural and functional parasitic components.

According to the present invention, a mutant microorganism is created that is incapable of growing under selection conditions in which the function of an absent or defective gene is required for viability. A target gene-complemented microorganism is obtained by complementing the mutant microorganism with a heterologous parasite gene encoding a parasite gene product that enables the mutant microorganism to grow in the selection conditions. In order to identify compounds that can function as inhibitors of the target gene, the target gene-complemented microorganism is exposed to compounds to be tested under the selection conditions for their ability to inhibit the target gene product. Due to the selection conditions, inhibition of the target gene product results in decreased viability of the target gene-complemented microorganism. Comparing the viability of the target gene-complemented microorganism exposed to the compound to the viability of the target gene-complemented microorganism in the absence of the compound can identify inhibitor compounds of the target gene. To identify a specific inhibitor of the parasitic target gene product which is also non-toxic to the non-complemented microorganism, the viability of the target gene-complemented microorganism after exposure to the compound in the presence of the selection conditions is compared to the viability of the microorganism in the presence of the compound but in the absence of the selection medium.

The first step in screening for antiparasitic compounds is the isolation of a parasite target gene that expresses a target enzyme that is essential for the development and survival of the parasite. The target enzyme is preferably a component of a metabolic pathway and catalyzes a rate-limiting step in the synthesis of compounds essential to the parasite. Once the target gene is identified, it may be cloned into a suitable plasmid which can be induced to express the parasitic enzyme from the target gene. This plasmid is then introduced into the mutant microorganism resulting in transformation of the mutant microorganism.

Preferably, the absence of, or defect in, the gene in the mutant microorganism does not prevent the organism's survival in normal conditions but renders it incapable of surviving in selection conditions, such as stress conditions. The transformation process results in the complementation of the defective or absent gene with the gene encoding the parasitic enzyme enabling the mutant microorganism to survive in selection conditions in a manner that may be indistinguishable from the wildtype.

As used herein, target gene-complemented microorganism, or recombinant microorganism, refers to a deficient or mutant microorganism which has been enabled to survive in selection conditions due to the introduction of a functioning homologous or heterologous gene, which is the target gene. Isolation of target gene-complemented microorganisms can be achieved in a single step by growing the complemented microorganisms in the selection conditions. Only complemented microorganisms will grow in the selection conditions. The target gene may remain in the vector used to tranforn the micro-organism or the target gene may become inserted in the chromosome of the microorganism.

The action of a compound on a target enzyme may be determined by comparing the viability of the two populations. As used herein, “comparing viability” means evaluating the relative conditions of microorganisms wherein death or retarded or impaired growth indicate low viability as compared to normal vigorous growth. By complementing the deficient microorganism with a gene that renders the microorganism viable in selection conditions, one can study its response to potential drugs in the presence of a selection condition. Agents that inhibit the growth and/or survival of the complemented microorganism when grown in selection conditions, but not in the absence of selection conditions, are potential antiparasitic compounds.

Selection condition, selection medium, and test condition describe conditions that will only allow growth of a microorganism which expresses a particular protein of interest. Selection conditions may include any condition that can discriminate between the expression or non-expression of a gene or genes. Non-limiting examples of such conditions include variations in osmolarity, temperature, chemicals, pressure or light. Selection conditions include stress conditions in which an organism experiences difficulty of growth due to the presence or absence of a factor or factors that perturb normal functioning of the organism. The organism can survive and grow in such stress conditions because it expresses a protein or proteins that work by lessening the effect of the perturbing factors and enabling normal functioning. In one embodiment of the invention, a stress condition is caused by high salt concentration that causes the exit of water from inside the organism so that normal processes inside the organism are adversely affected. One way that such osmotic stress can be overcome is by producing an osmoprotectant such as glycerol, mannitol, trehalose or any other polyol. Production of osmoprotectants can be achieved by the induction of a specific gene or genes in response to high salt concentration. Lessening the effect of the perturbing factors may also be achieved by non-expression of a gene or genes.

The present invention is directed to a screening method for identifying compounds having antiparasitic acitivity. The compound identified by the method may be any compound having the desired properties, including large or small organic or inorganic molecules, biological polymers such as DNA, RNA, PNA, combinations or modifications thereof, polypeptides, antisense molecules and the like. Preferably, the compound inhibits the activity of the enzyme expressed by the target gene. The compound may also inhibit the expression of the target gene itself, thereby inhibiting the amount of expressed enzyme.

Another aspect of the invention is that the target gene-complemented microorganism requires the product of the target gene to survive in test conditions. Any compound that inhibits growth of the target gene-complemented microorganism in test conditions could be doing so by inhibiting the target enzyme or by inhibiting any other essential enzyme. Thus, determining whether a compound inhibits growth or survival of the target gene-complemented microorganism in conditions in which the function of the parasite gene product is not required for viability can be used to identify compounds that specifically and selectively inhibit the target gene product.

In another aspect of the invention, the selection conditions are designed so that mutant microorganisms can grow if they express the target gene. If the selection forces are removed from the selection conditions, both the non-complemented microorganism and the target gene-complemented microorganism will survive since expression of the target gene is not essential. Thus, by changing the composition of the selection medium, conditions in which the function of the parasite gene product is not required for viability can be used to identify compounds that specifically and selectively inhibit the target gene product. Compounds that selectively inhibit the target gene preferably include compounds that cause reduced viability of target gene-complemented microorganisms in selection medium but do not cause reduce viability in the absence of the selection medium.

One embodiment of the invention provides for a method for screening for inhibitors of the product of a target gene derived from the protozoal phylum Apicomplexa. The target gene can be used to complement a mutant microorganism that lacks a functional homologous or heterologous gene. A suitable example of a mutant microorganism is a mutant Saccharomyces cerevisiae. The principal osmolyte in yeast is glycerol, although other polyols such as trehalose, arabinose and mannitol are produced in yeast and in some fungi. Disruption of the gpd1 gene encoding glycerol-3-phosphate dehydrogenase in S. cerevisiae results in a mutant strain lacking the ability to produce glycerol in response to osmotic stress. Because mannitol can substitute for gycerol in yeast, the osmotolerance of the mutant S. cerevisiae can be restored when the mutant organism is transformed with a gene encoding mannitol-1-phosphate dehydrogenase (m1pdh).

In a preferred embodiment of the invention, a screening method is provided to screen for inhibitors of M1PDH of organisms in the phylum Apicomplexa. In one example, mutant S. cerevisiae, defective in gpd1, is complemented with m1pdh from Eimeria tenella. Functional expression of the E. tenella gene encoding m1pdh in this mutant strain is able to restore osmotolerance. This dependence on the target gene for osmoregulation can be used as a means of selection. Thus, the gene complemented microorganism that depends for survival on the function of a parasite enzyme provides a tool for screening compounds that inhibit the parasite enzyme.

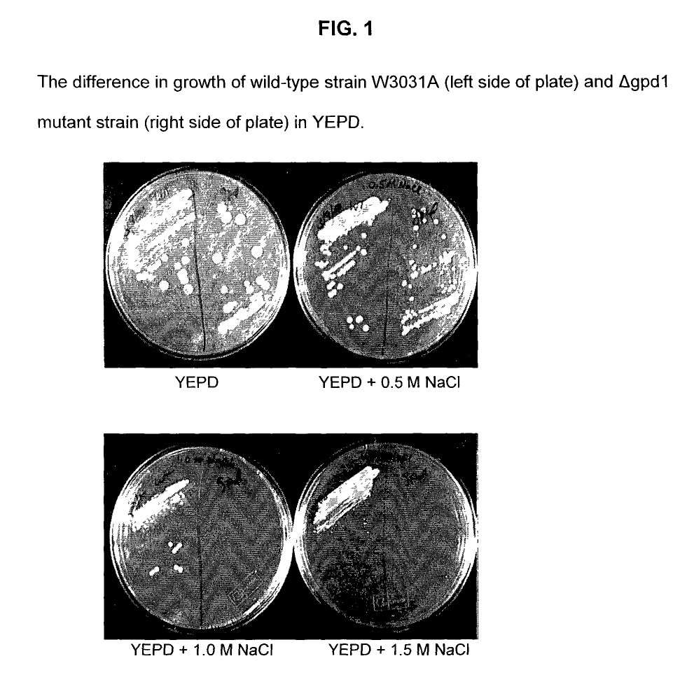

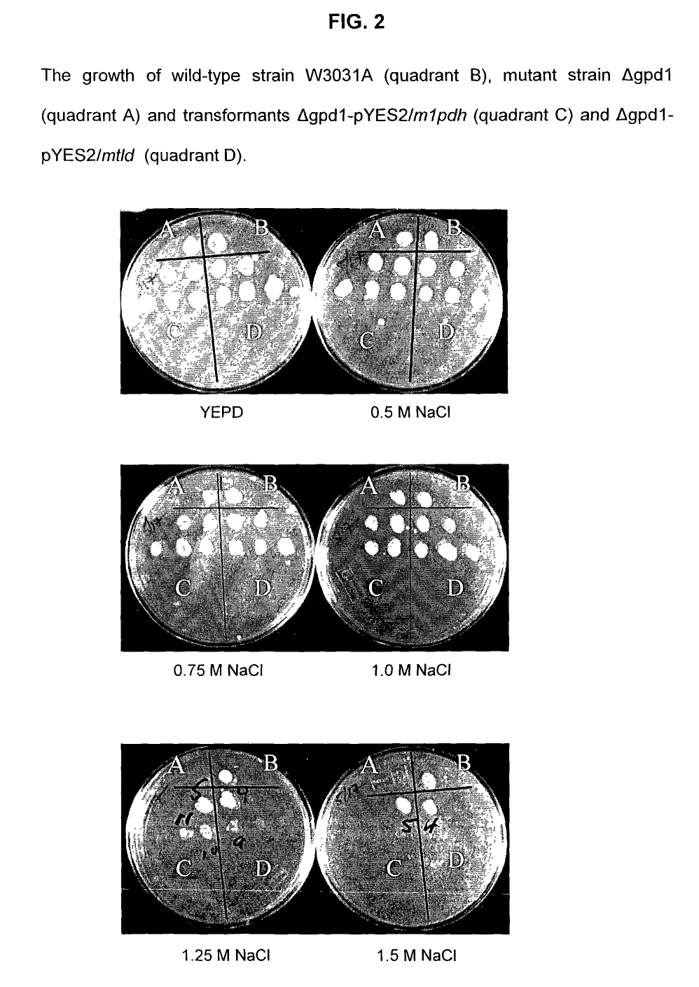

Using the m1pdh gene from Eimeria tenella, the ability to synthesize mannitol was shown to be directly effective in the development of osmotolerance in a yeast strain lacking the gpd1 gene. Referring now to FIG. 1, mutant yeast of the strain W303-1A lacking gdp1 (“Δgpd1”) is plated next to wild type W303-1A on YEPD in increasing concentrations of NaCl. Mutant strain Δgpd1 cannot grow when the NaCl concentration exceeds 1M. To restore osmotolerance of the mutant strain, the strain was transformed with a plasmid containing E. tenella m1pdh. As shown in FIG. 2, the gene-complemented transformant, Δgpd1/pYES2-m1pdh, is able to grow in salt concentrations of 1.5M. This was confirmed by using the gene encoding E. coli mannitol-1 phosphate dehydrogenease, mt1D, in place of the E. tenella m1pdh gene in a similarly constructed transformant, Δgpd1-pYES2/mt1D, as a positive control.

E. tenella m1pdh operates preferentially to produce mannitol-1-phosphate from fructose-6-phosphate resulting in the accumulation rather than the utilization of mannitol. A screening strategy must account for the directionality of the enzyme. The present invention uses the selective advantage gained by a microorganism due to the accumulation of mannitol. Thus, complementation of the yeast gpd1 gene with the E. tenella m1pdh cDNA should result in the production of mannitol and increased osmotolerance.

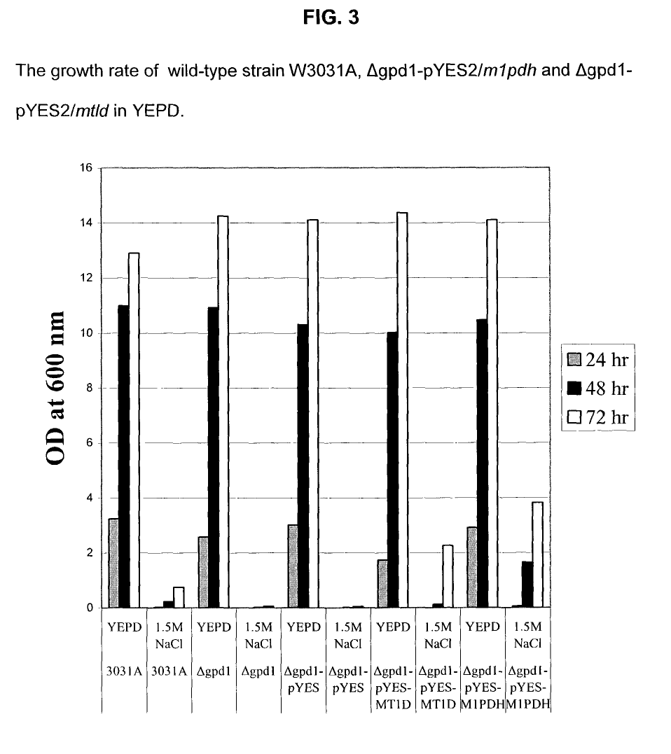

High level expresssion of E. tenella m1pdh is toxic to host, especially mammalian cells and E. coli, as shown in the literature. Therefore, a step-wise selection may be used to obtain complemented strains. The phenotype of S. cerevisiae transformants maintained on minimal yeast media supplemented with galactose and raffinose is monitored by plating cells at 24 hour intervals on YEPD media containing various concentrations of NaCl. FIG. 3 shows growth over a 72 hr period. It has been found that the most efficient induction period is between 48 and 72 hours. The step-wise transfer of colonies growing on lower to higher salt concentrations (0.5 to 2 M) resulted in two clones that grew robustly in 1.5 M NaCl as shown in FIG. 2. Comparison of growth rates in medium containing 1.5 M NaCl in tube cultures shows a very distinct difference between strains transformed with mt1D or m1pdh and those transformed with either vector only or no plasmid.

These differences in growth rates are also evident in a 96-well plate format, suitable for high through-put (HTS) screening. Cultures of strains transformed with mt1D or m1pdh can grow detectably in medium containing 1.5 M NaCl, as did the wild-type strain. Growth in medium containing lower concentrations of NaCl is not dependent on the functional expression of the parasite enzyme. In this system, cells are first tested for sensitivity to random chemicals in medium containing 1.5 M NaCl. In this environment, the cells require the function of the parasite m1pdh gene product for survival. Compounds that inhibit growth or survival of the recombinant yeast in 1.5 M NaCl are then retested in medium containing no additional NaCl. Compounds that are no longer toxic are candidate m1pdh inhibitors as they are only active when the cell is dependent upon the operation of the parasite enzyme.

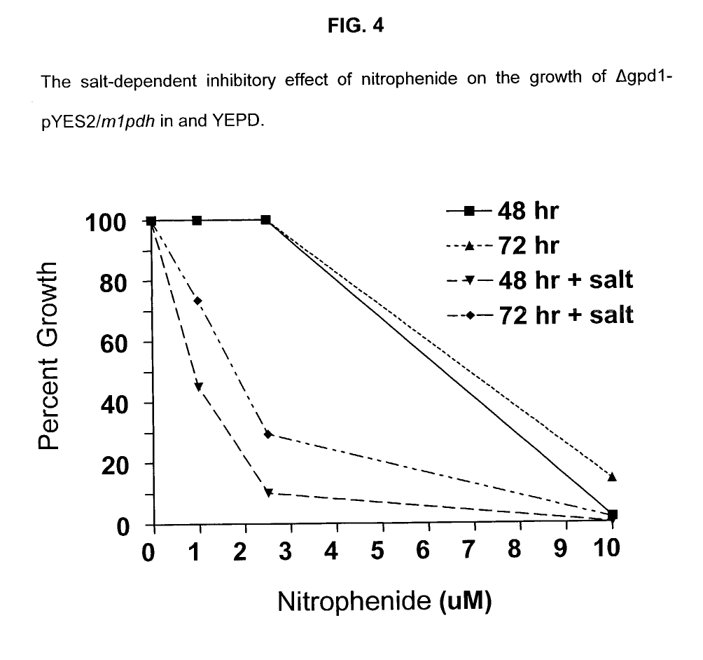

In one embodiment, the method of the invention can be used to screen for novel inhibitors of coccidial M1PDH. This is demonstrated by using nitrophenide, a well known inhibitor of coccidial M1PDH. As shown in FIG. 4., growth of the gene complemented S. cerevisiae expressing m1pdh is dramatically suppressed by nitrophenide in medium containing 1.5 M NaCl, conditions in which a functional M1PDH is needed for survival. The toxicity of this compound is reduced when cells are grown in medium to which no additional NaCl has been added indicating that suppression of growth in the presence of 1.5 M NaCl is due to specific inhibition of M1PDH.

The methods of the present invention do not require that all the genes coding for all the essential enzymes of the parasite organism be known or be expressed within a mutant microbial strain. All that is required is one essential parasite gene product which when inhibited will prevent survival of the parasite. Further, the essential parasite gene must be able to complement the mutant microorganism enabling it to survive in stress conditions. Thus, the methods of the present invention are useful in screening for compounds which inhibit the activity of a parasite enzyme. The screening method includes exposing to a compound a parasite target gene-complemented microorganism growing in a selection medium that inhibits the viability of target gene-complemented microorganisms.

When the selection forces are removed from the growth medium, both the non-complemented microorganism and the target gene-complemented microorganism will survive since expression of the target gene is not essential. Thus, by changing the composition of the selection medium, conditions in which the function of the parasite gene product is not required for viability can be used to identify compounds that specifically and selectively inhibit the target gene product. Compounds that selectively inhibit the target gene product preferably include compounds that cause reduced viability of target gene-complemented microorganisms in selection medium but do not cause reduce viability in the absence of the selection medium.

The methods of this invention are useful for screening compounds as inhibitors of M1PDH in various species of organisms having a mannitol cycle including but not limited to species in the phylum Apicomplexa, for example, Eimeria tenella, Cryptosporidium parvum, Toxoplasma gondii, Plasmodium spp. and Isospora spp. Other organisms may include Alternaria alternata, Echerichia coli, Campylobacter jejuni, Salmonella enteritidis, Shigella sonnei, Listeria monocytogenes, Pseudomonas aeruginosa, Cryptococcus neoformans as well as the ascomycetous and basidiomycetous ectomycorrhizal fungi. The methods of this invention can also be useful to identify compounds that inhibit other enzymes in the synthesis of mannitol such as mannitol-1-phosphatase, mannitol dehydrogenase and hexokinase. These enzymes have been detected in multiple species of the genus Eimeria that infect poultry, suggesting that inhibitors of M1PDH should be useful in controlling poultry coccidiosis as this genus is present and pathogenic in poultry-raising operations. In addition, those skilled in the art will understand that the invention may be used to screen for inhibitors of any parasite enzyme for which the gene can be inserted into a mutant microorganism that depends up on the function of the parasite gene for survival

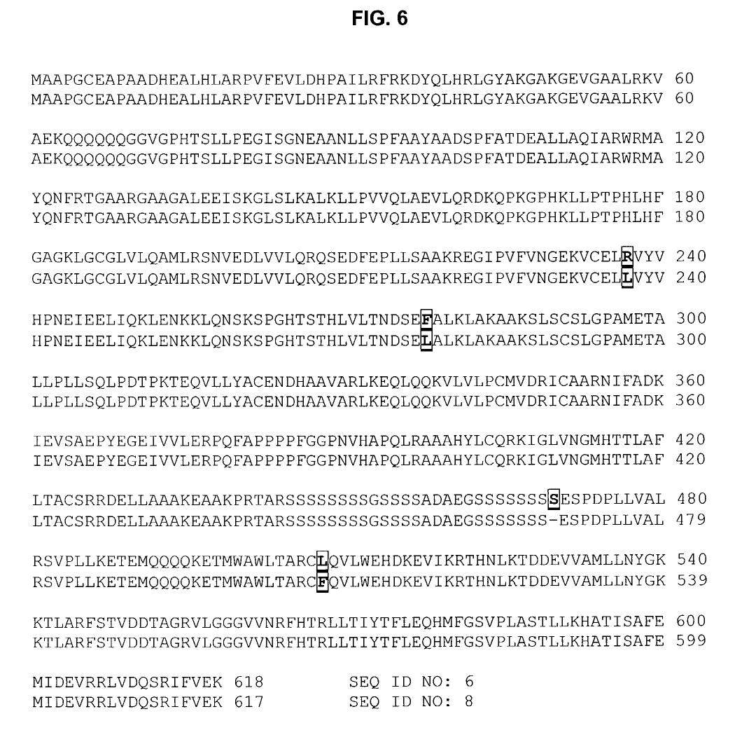

In another embodiment, the invention is directed to an amino acid sequence of a functional parasitic M1PDH. FIG. 6 shows the Eimeria tenella M1PDH amino acid sequence of the present invention (SEQ ID NO: 6) compared to the sequence of M1PDH isolated from unsporulated Eimeria tenella oocysts described by Liberator et al., 1997 in GenBank as protein ID AAD02688 (SEQ ID NO: 8). The amino acid sequence of present invention was deduced from the M1PDH DNA sequence isolated from sporulated oocysts of Eimeria tenella (SEQ ID NO: 5). Four differences between the amino acid sequence of the invention and the sequence of Liberator et al., are apparent: position 237 of the sequence of the invention provides for arginine instead of leucine; position 278 provides for phenylalanine instead of leucine; position 470 includes an extra serine residue; and position 508 provides for leucine instead of phenylalanine. It is expected these differences are due to E. tenella strain variation.

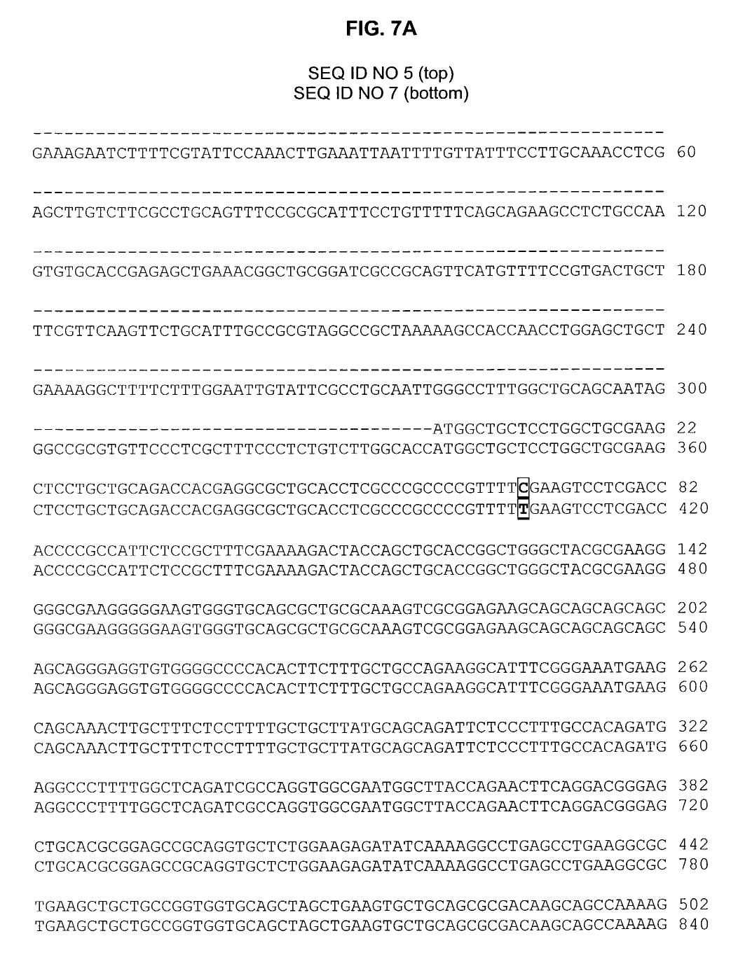

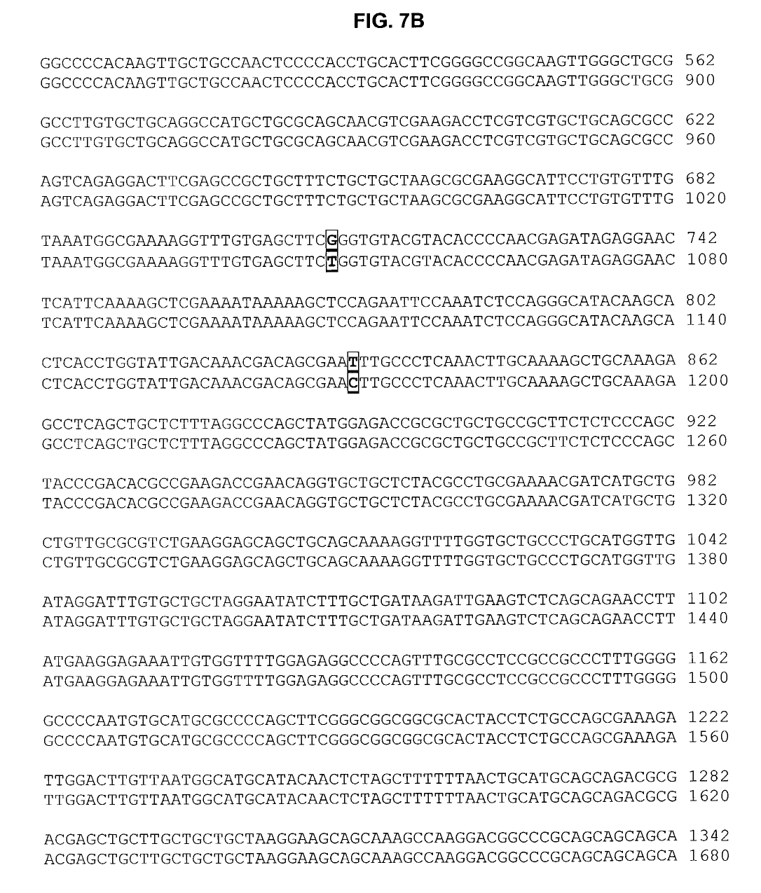

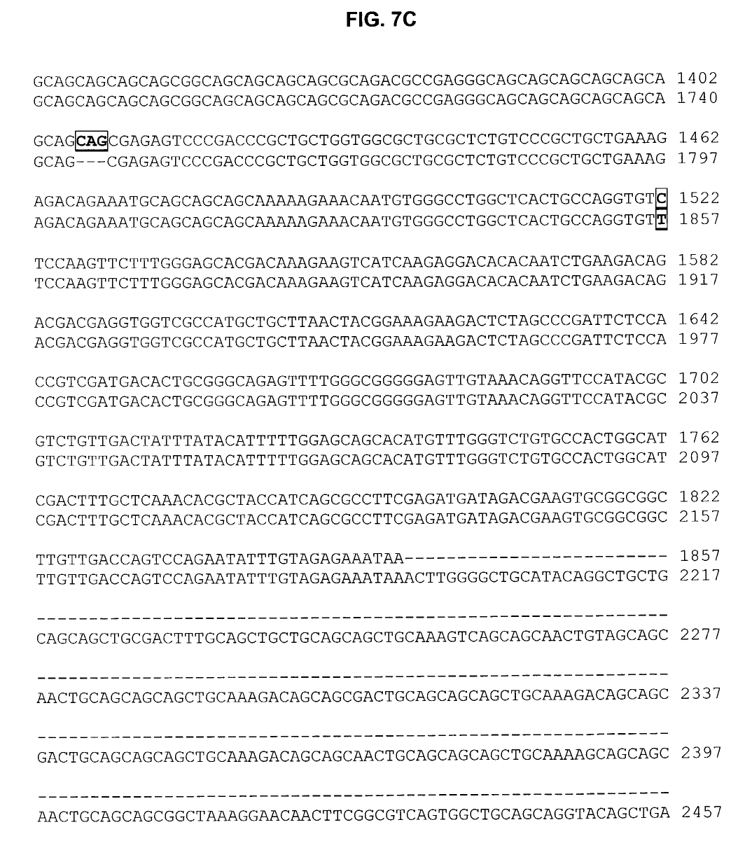

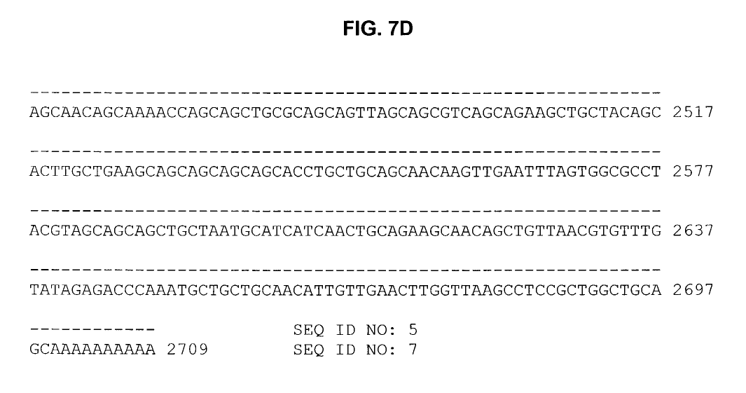

In another embodiment, the invention is directed to a nucleic acid sequence encoding the amino acid sequence of SEQ ID NO: 5. FIGS. 7A-D show one such sequence which was obtained from the m1pdh gene isolated from sporulated oocysts as further described in the Examples [SEQ ID NO: 5]. This sequence is compared to the sequence described by Liberator et al. 1997, Genbank Accession No. AF055716 [SEQ ID NO. 7]. Numerous differences between the polynucleotide sequence of the present invention and Liberator et al. are apparent. However, only four of those differences encode different amino acids which result in the differences between the polypeptide sequence of the present invention [SEQ ID NO: 6] and the polypeptide sequence of Liberator et al. [SEQ ID NO. 8].

One skilled in the art would understand that numerous modifications may be made to the nucleic acid sequence of the present invention that, when expressed, would not alter the sequence of the M1PDH of the present invention. Silent mutations may be established to allow for better expression in a particular organism or for other reasons known to those skilled in the art. Thus, the present invention encompasses any nucleic acid sequence, which due to the degeneracy of the genetic code, encodes the amino acid sequence of SEQ ID NO. 8.

### EXAMPLES

### Example 1

Preparation of Growth Media

Suitable media for growing yeast and general culture methods are those generally used and described in many widely available manuals and published manuscripts (see, for example, Klein and Roof, 1988). These materials and procedures are well known to those skilled in the art. Many kinds of yeast media can be used for such experiments. One example is YEPD: 10 g yeast extract+20 g peptone+20 g glucose+20 g agar in one liter of water; autoclaved for sterility; agar may be left out to obtain a liquid media instead. A second example is YMM: to 500 ml of water, add 20 g glucose+7 g nitrogen base minus amino acids+30 mg of either uracil, adenine, tryptophan, leucine, histidine, or lysine; filter to sterilize; add 20 g agar and water to make one liter for solid media; autoclave for 15 min; amino acids are added as appropriate for the auxotrophic markers on the vectors used for yeast transformations. In some cases, the glucose component of these media can be replaced by other sugars. For instance, glucose (20 g) can be replaced with a combination of galactose (20 g) and raffinose (40 g). This combination allows robust growth while inducing expression of genes under the control of the gal-1 promoter, and is well known to those skilled in the art.

### Example 2

Yeast Strains and Preparation of Plasmids

Two yeast strains, W303-1A (ATCC 208353 and 208352) and W303-1AΔgdp1 were obtained from Prof. Lennart Adler (Goteberg, Sweden) and are described in Ansell et al., 1997 and Albertyn et al., 1994. W303-1AΔgpd1 (“Δgpd1”) is a strain from which the gene that encodes glycerol-3-phosphate dehydrogenase (gdp1) has been deleted.

To prepare Δgpd1, a 2.5 kb PstI fragment from pCS19 (ATCC 77409) yeast genomic library containing the gdp1 coding region may be subcloned into pUC19 (Invitrogen, San Diego, Calif.). Two primers may be used to amplify the whole plasmid except for the gpd1 coding region by polymerase chain reaction (PCR): 35 cycles of 1 min at 96° C., 1 min at 51° C., and 3 min at 72° C. Primer 1 is 5′-AGAAGTTAGTACAGGCCGTC-3′ (SEQ ID NO: 1) and is complementary to the 3′ end of the coding region, leaving the BglII site which is located 3 codons upstream of the stop codon within the amplified sequence. Primer 2 is 5′-GAAGATCTTCAATATTTGTGTTTGTGGAGGG-3′ (SEQ ID NO: 2) and is complementary to a region just upstream of the ATG start codon and introduces a BglII site. The amplification product can be digested with BglII and ligated to give a circular plasmid having a BglII site instead of the gdp1 coding region (pUCgpd1Δ). This procedure should delete 1.17 kb of DNA. The TRP1 gene can be cut out of plasmid YDpW on a 0.86 kb BamHI fragment and inserted into the BglII site of pUCgpd1Δ to give plasmid pUCgpd1Δ::TRP1. This plasmid can be cut with PstI and transformed into yeast cells to delete the gdp1 gene from the genome according to the method described by Rothstein (1983). The deletion can be confirmed in tryptophan prototrophic transformats by Southern blot analysis using EcoRI-digested chromosomal DNA and the plasmid UCgpd1Δ or a PstI-BglII fragment of the gdp1 coding region as probe.

The Δgpd1 strain is deficient in managing osmotic stress and grows poorly or not at all in high salt concentrations (Ansell et al., 1997). Plating tests of the wild type strain, W303-1A and Δgpd1 on solid YEPD medium containing various NaCl concentrations confirmed the osmosensitive phenotype of Δgpd1 as is shown in FIG. 1. W303-1A grows well on medium containing concentrations of salt as high as 1.5 M, while Δgpd1 does not grow on YEPD plates containing 1.0 M or 1.5 M NaCl. The inclusion of 2.0 M NaCl in YEPD prevents growth of the wild-type strain W303-1A (not shown).

To prepare a gene complemented microorganism expressing E. tenella m1pdh, the poly(A)+mRNA coding for E. tenella m1pdh was isolated from sporulated oocysts using standard protocols (Sambrook et al., 1989). This mRNA was used as a template to synthesize a cDNA using Superscript II Rnase H reverse transcriptase according to the manufacturer's instructions (GibcoBRL, Gaithersburg, Md.). Polymerase chain reaction (PCR) was used to amply the gene coding for E. tenella m1pdh using the synthesized cDNA as templates and primers designed based on the sequence data reported by Liberator et al. 1997 (GenBank AF055716). Primers for the PCR were SCN64 (sense primer): 5′-CTCTGTCTTGGCACCATGGCTGCTCCTGGC-3′ (SEQ ID NO: 3) and SCN65 (antisense): 5′-TGCTGCAGCAGCCTGTATGCAGCCCCAAGT-3′ (SEQ ID NO: 4). PCR reactions contained 0.4 uM each primer, 200 uM dNTPs, 1.15 mM Mg(OAc)₂ and 4 units of rTth DNA polymerase, XL (Perkin Elmer, Branchburg, N.J.) in a final volume of 100 ul. The PCR conditions were 40 cycles at 94° C. for 30 sec, 70° C. for 5 min, followed by 72° C. for 7 min using a Perkin Elmer 9600 temperature cycler. A TA cloning kit (Invitrogen, San Diego, Calif.) was used to clone PCR products into the vector pCR2.1. Plasmids were propagated in E. coli strain SURE2 (Stratagene, La Jolla, Calif.). Colony hybridization was performed using the Genius System (Boehringer Mannheim, Indianapolis, Ind.) according to the kit instructions using a digoxigenin-labeled m1pdh PCR product as the probe. Hybridizing colonies were isolated and purified using a Qiagen column (Valencia, Calif.). Clones were identified by restriction endonuclease analysis using EcoRI (New England Biolabs, Beverly, Mass.). Nucleotide sequence [SEQ ID NO 5] was determined by automated sequencing on an ABI 377 instrument (PE Applied Biosystems, Inc., Foster City, Calif.) using reagents and conditions specified by the manufacturer. Sequencing oligonucleotides primers were purchased from Genosys.

The cDNA was also cloned into the commercially available vector pCRII (Invitrogen, Carlsbad, Calif.) and a BstX1 fragment from the pCRII containing the cDNA was sub-cloned into the BstX1 site of pYES2, a commercially available yeast shuttle vector with a gal-1 promoter (Invitrogen, Carlsbad, Calif.). The resulting plasmid was termed pYES2/m1pdh.

Southern hybridization analysis for characterization of plasmid in the transformed yeast was performed according to standard techniques as previously described in Klein et al. (1991). The probe used for the Southern hybridization analysis was derived from an EcoRI digest of the pCRII plasmid containing the cDNA encoding E. tenella m1pdh. There is one internal EcoRI site in this insert, so the digest produces two fragments, both of which were isolated, pooled and labelled.

To prepare the control recombinant organism, Echerichia coli MC11 gene mt1D encoding mannitol-1-phosphate dehydrogenase (GenBank X51359) was obtained from Brian Wong (Yale University). This gene served as a positive control as described by Chaturvedi et al. (1997). The procedure for the isolation and cloning of mtlD into plasmid pYES2 was as generally described by Chaturvedi et al. (1997) and Jiang et al. (1990).

### Example 3

Yeast Transformations

The Δgpd1 strain was transformed with pYES2, pYES2/mt1D, or pYES21/m1pdh using a LiAc method adapted from well-established standard methods such as Bartel et al., (1993). Transformants are selected on agar YMM plates and grown in YMM liquid medium with raffinose and galactose as carbon sources to increase expression from the gal-1 promoter. Samples are plated at 24 hour intervals on agar plates containing YEPD medium supplemented with 0.5, 1.0 and 1.5 M NaCl. Controls in growth experiments included the wild-type strain W303-1A, untransformed Δgpd1, and Δgpd1transformed with pYES2 only (plasmid without any insert).

### Example 4

Liquid Assay

Transformants growing on high salt plates were picked and grown in 18×150 mm glass culture tubes containing 10 ml liquid YEPD medium or liquid YEPD medium with 1.5 M NaCl. These tubes were inoculated with approximately 10⁵ cells and cultured at 30° C. on a roller bottle. Growth rates were recorded in terms of optical density values at various times after inoculation. Growth rates were also monitored in 96-well plates using the indicator dye Alamar Blue (BioSource International, Camarillo, Calif.) as previously described by Klein et al., (1997b). In 96-well plate experiments, each well contained approximately 10⁴ cells (in 20 ul medium) inoculated into 180 ul liquid YEPD medium or liquid YEPD medium containing 1.5 M NaCl. These plates were then incubated at 30° C. for 24 or 48 hours. At that time, 20 ul Alamar Blue solution was added to each well and the color allowed to develop for 5-30 minutes. Color changes in each well were noted after visual inspection and recorded.

### Example 5

Selective Inhibition of Eimeria tenella m1pdh

Nitrophenide, a known inhibitor of MIPDH (Schmatz 1997), was used to validate the assay. The Δgpd1transformed with pYES21/m1pdh was grown in liquid YEPD medium, either supplemented with 1.5 M NaCal or not and containing one of 3 concentrations of nitrophenide (Aldrich Chemical Co.) dissolved in DMSO. Experiments were conducted in glass culture tubes prepared and processed as described above. Growth in nitrophenide-containing media was compared to growth in identical medium lacking nitrophenide by measuring and recording changes at OD 600 nm after 24, 48 or 72 hours in culture as described above.

### Example 6

Measurement of Mannitol Biosynthesis

Mannitol production was monitored as described by Shen et al., (1999). Briefly, yeast strains were grown in liquid YEPD or YMM media. YEPD medium was supplemented with 1.5 M NaCl. Cultures were inoculated with W303-1A or Δgpd1 transformed with pYES2, pYES2/mt1D or pYES21/m1pdh. Cultures in YEPD-1.5 M NaCl were allowed to grow to a final OD of approximately 6. Cells were collected by centrifugation and concentrated to approximately 4×10⁸ cells in 2 ml. After centrifugation of this suspension, the cell pellets were suspended in YMM-glucose. This suspension was centrifuged and the supernatant removed. The cell pellet was suspended in 500 ul extraction solvent (chloroform:ethanol:water, 3:5:1, v/v/v) and vortexed for 10 minutes. Water (500 ul) was added to this suspension and the mixture was centrifuged at 12,000 rpm for 10 minutes (Tomy refrigerated microfuge). The aqueous layer (approximately 600 ul) was removed and passed through a small Amberlite/Dowex column. The column was washed twice with 200 ul ethanol/water (1:1) and the eluates pooled and vacuum dried. The solid material was then dissolved in 250 ul double-distilled water and passed through a 0.2 micron Acrodisc nylon filter.

The monosaccharide composition of the aqueous solution was determined by High Pressure Anion Exchange Chromatography (HPAEC; Hardy 1988), using a Dionex DX-500 liquid chromatograph equipped with pulsed amperometric detection. Samples (15 ul) were injected onto a CarboPac PA1 analytical column (4.6×250 mm) and eluted isocratically in 16 mM NaOH at a flow rate of 1 ml/min. Chromatographic peaks were identified and quantified by comparing the retention times and the integrated peak areas to reference monosaccharides using Dionex PeakNet 6.0 software.

A similar protocol was followed for cells grown in YMM-galactose/raffinose. When cell density approaches 0.4 OD units, cultures were centrifuged at low speed, the supernatant removed, and the cells washed in YMM. These cells were then suspended in YMM-galactose/raffinose containing 1.5 M NaCl and allowed to grow until the OD reached approximately 0.6 units. At this point, the cells were harvested by low-speed centrifugation and processed as described above.

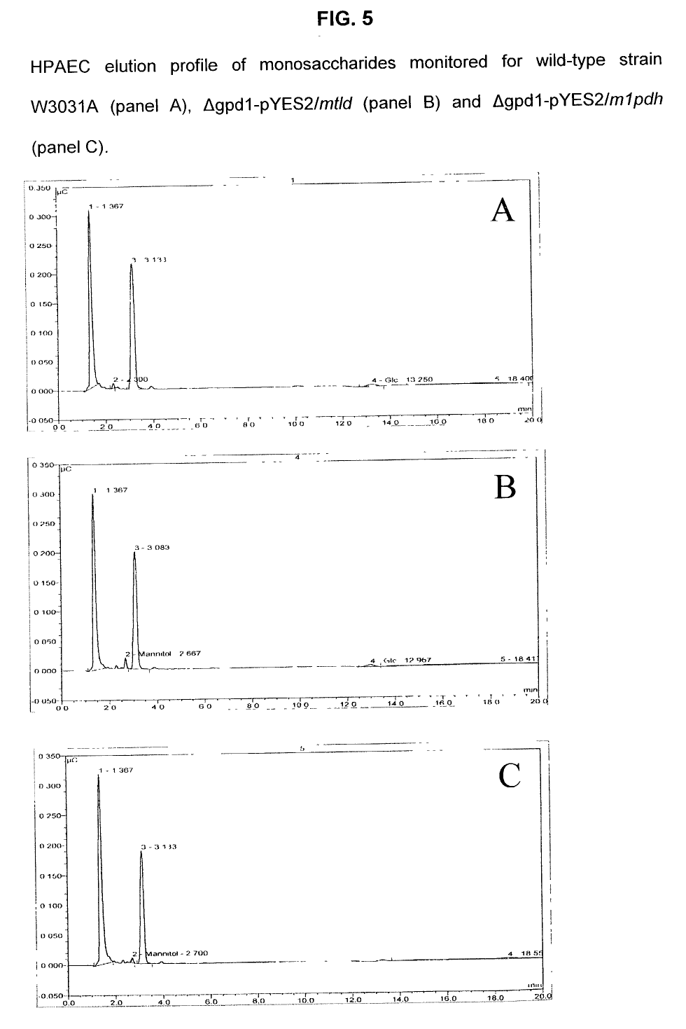

Mannitol production was monitored in yeast strains W303-1A, Δgpd1-pYES2/mtlD and Δgpd1-pYES2/m1pdh grown in YEPD with 1.5 M NaCl or in YMM with raffinose-galactose. Cells grew more robustly in YEPD with 1.5 M NaCl and mannitol production was more readily apparent in these cultures as shown in FIG. 5. Growing cells also produced trehalose (peak at 3.1 min) as an oxidoprotectant, which was more prominent than mannitol as can be see in FIG. 5. Under the protocol used, approximately 1 unmol mannitol per 10⁸ pYES2/m1pdh cells was detected when grown in YEPD with 1.5 M NaCl. Table 1 shows the of the amount of mannitol, glucose and trehalose produced by wild-type strain W3031A, and transformants Δgpd1-pYES2/mt1D and Δgpd1-pYES2/m1pdh. This data was derived from FIG. 5.

TABLE 1

| | Ret. Time | | Area | Rel. Area | Amount |
|---|---|---|---|---|---|
| Strain | (mm) | Peak Name | (uC × min) | (%) | (pmol) |
| W303-1A | 1.37 | | 0.039 | 45.47 | 0.039 |
| | 2.3 | | 0.001 | 0.76 | 0.001 |
| | 3.13 | Trehalose | 0.042 | 48.59 | 0.042 |
| | 13.25 | Glucose | 0.002 | 1.96 | n.a. |
| | 18.4 | | 0.003 | 3.21 | 0.003 |
| Δgpd1-pYES2/mtlD | 1.37 | | 0.040 | 48.52 | 0.040 |
| | 2.67 | Mannitol | 0.002 | 1.98 | 104.289 |
| | 3.08 | Trehalose | 0.035 | 41.97 | 0.035 |
| | 12.97 | Glucose | 0.001 | 1.49 | n.a. |
| | 18.42 | | 0.005 | 6.04 | 0.005 |
| Δgpd1-pYES2/m1pdh | 1.37 | | 0.045 | 55.24 | 0.045 |
| | 2.70 | Mannitol | 0.001 | 0.88 | 45.894 |
| | 3.13 | Trehalose | 0.031 | 38.05 | 0.031 |
| | 18.55 | | 0.005 | 5.83 | 0.005 |

### Example 7

Selection of Yeast Clones

After 48-72 hours in liquid YMM supplemented with galactose and raffinose, all transformants grew well when streaked on YEPD plates containing up to 1.0 M NaCl. Phenotypic discrimination was evident at higher concentrations of salt; at 1.5 M, only the wild-type strain and Δgpd1 transformed with m1pdh or mt1D grew as shown in FIG. 2. Two clones from each category that grow robustly on 1.5 M NaCl in YEPD were selected for further characterization. A set of 5 test groups was selected for growth and inhibition experiments, including:

1. wild-type yeast (W303-1A)

2. Δgpd1 (untransformed)

3. Δgpd1-pYES2

4. Δgpd1-pYES2/mt1D

5. Δgpd1-pYES2/m1pdh

### Example 8

Growth and Inhibition Studies

Test groups of each sample organism were inoculated into liquid cultures of YEPD and YEPD with 1.5 M NaCl. Rate of growth was estimated by recording OD 600 values at 24 hr intervals. All strains grew equally well in YEPD, but growth rates in YEPD with 1.5 M NaCl were distinctly different between transformants expressing mt1D or m1pdh and controls as shown in FIG. 3. W303-1A grew less well in high salt than Δgpd1-pYES2/mt1D or Δgpd1-pYES2/m1pdh. Δgpd1 and Δgpd1-pYES2 did not grow in medium containing 1.5 M NaCl.

Nitrophenide was dissolved in DMSO and added to cultures at final concentrations of 1 μM, 2.5 μM or 10 μM. DMSO alone was also tested. Little toxicity was apparent for DMSO or for nitrophenide in YEPD except at 10 μM, a concentration at which slowing of the rate of growth of Δgpd1-pYES2/mt1D and Δgpd1-pYES2/MIPDH was observed as shown in FIG. 4. However, in YEPD with 1.5 M NaCl, nitrophenide was toxic to Δgpd1-pYES2/mt1D and Δgpd1-pYES2/MIPDH at concentrations as low as 2.5 uM and abolishes growth at 10 uM as shown in FIG. 4. Nitrophenide was also toxic to W303-1A under these conditions, but this strain grew more slowly than either of the transformants under salt pressure. The difference in rate of growth made comparisons between strains difficult.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims.

### REFERENCES

Albertyn, J., Hohmann, S., Thevelein, J. M., Prior, B. A. “GPD1, which encodes glycerol-3-phosphate dehydrogenase, is essential for growth under osmotic stress in Saccharomyces cerevisiae, and its expression is regulated by the high-osmolarity glycerol response pathway.” Mol. Cell Biol. 1994; 14:4135-44

Allocco, J. J., Profous-Jucheika, H., Myers, R. W., Nare, B., Schmatz, D. M., “Biosynthesis and catabolism of mannitol is developmentally regulated in the protozoan parasite, Eimeria tenella.” J. Parasitol. 1999; 85:167-173.

Allocco, J. J., Nare, B., Myers, R. W., Feiglin, M., Schmatz, D. M., Profous-Juchelka, H. “Nitrophenide (Megasul™) blocks Eimeria tenella development by inhibiting the mannitol cycle enzyme mannitol- 1-phosphate dehydrogenase.” J. Parasitol. 2001; 87:1441-1448.

Alakhov, V., Pietrzynski, G., Kabanov, A. “Combinatorial approaches to formulation development.” Curr. Opin. Drug Discov. Devel. 2001; 4:493-501

Ansell, R., Granath, K., Hohmann, S., Thevelein, J. M., Adler, L. “The two isoenzymes for yeast NAD(+)-dependent glycerol-3-phosphate dehydrogenase encoded by GPD1 and GPD2 have distinct roles in osmoadaptation and redox regulation.” EMBO J. 1997; 16:2179-2187.

Bartel, P. L., Chien, C.-T., Stemglanz, R., Fields, S., “Using the two-hybrid system to detect protein-protein interactions. In: Cellular Interactions and Development: A Proactical Approach” Hartley, D. A., editor, Oxford University Press, Ox ford, 1993. pp. 153-179.

Björkqvist, S., Ansell, R., Adler, L., Liden, G. “Physiological response to anaerobicity of glycerol-3-phosphate dehydrogenase mutants of Saccharomyces cerevisiae.” Appl. Env. Microbiol. 1997; 63:128-132.

Chapman, H. D., “Biochemical, genetic and applied aspects of drug resistance in Eimeria parasites of the fowl.” Avian Pathol. 1997; 26:221-244.

Chaturvedi, V., Bartiss, A., Wong, B., “Expression of bacterial mt1D in Saccharomyces cerevisiae results in mannitol synthesis and protects a glycerol-defective mutant from high-salt and oxidative stress.” J. Bacteriol. 1997; 179:157-162.

Coombs, G. H., “Biochemical peculiarities and drug targets in Cryptosporidium parvum: lessons from other coccidian parasites.” Parasitol. Today 1999; 15:333-338.

Hardy, M. R., Townsend, R. R., Lee, Y. C. “Monosaccharide analysis of glycoconjugates by anion exchange chromatography with pulsed amperometric detection.” Anal. Biochem. 1988; 170:54-62.

Jiang, W., Wu, L. F., Tomich, J., Saier, M. H. Jr, Niehaus, W. G. Corrected sequence of the mannitol (mtl) operon in Escherichia coli.” Mol. Microbiol. 1990; 4:2003-6

Klein, R. D., Favreau, M. A. “A DNA fragment containing the ADE2 gene from Schwanniomyces occidentalis can be maintained as an extrachromosomal element.” Gene 1991; 97:183-189.

Klein, R. D., Favreau, M. A., Alexander-Bowman, S. J., Nulf, S. C., Vanover, L., Winterrowd, C. A., Yarlett, N., Martinez, M., Keithly, J. S., Zantello, M. R., Thomas, E. M., Geary, T. G. “Haemonchus contortus: cloning and functional expression of a cDNA encoding ornithine decarboxylase and development of a screen for inhibitors.” Exp. Parasitol. 1997; 87:187-194.

Klein, R.D., and Roof, L. L. “Cloning of the orotidine 5′-phosphate decarboxylase (ODC) gene of Schwanniomyces occidentalis by complementation of the ura3 mutation in S. cerevisiae.” Current Genetics 1988; 13: 29-39

Lee, C. A., Saier, M. H. Jr. “Use of cloned mtl genes of Escherichia coli to introduce mtl deletion mutations into the chromosome.” J. Bacteriol. 1983; 153:685-92

Liberator, P. A., Anderson, J., Hozza, M., Profous-Juchelka, H., Sardana, M., Schmatz, D., Myers, R. “Molecular cloning and expression of mannitol-1-phosphate dehydrogenase from Eimeria tenella-association of recombinant m1pdh with heterologous 14-3-3 proteins.” Keystone Symposium on Molecular and Cellular Biology of Apicomplexan Protozoa, Park City, Utah, 1997; Abstract 211.

Myers, R., Liberator, P., Sardana, M., Aikawa, M., Allocco, J., Anderson, J., Wood, T., Fujioka, H., Schmatz, D. “14-3-3 Protein regulation of mannitol metabolism in Eimeria tenella via inhibition of mannitol- 1-phosphate dehydrogenase.” Keystone Symposium on Molecular and Cellular Biology of Apicomplexan Protozoa, Park City, Utah., 1997; Supplemental Abstracts.

Sambrook, Fritsch and Maniatis, Molecular Cloning: A Laboratory Manual, 2^{nd} ed., Cold Spring Harbor Laboratory Press, Cold Spring Harbor, NY, 1989.

Schmatz, D. M. “The mannitol cycle in Eimeria.” Parasitol. 1997;114:S81-S89.

Shen, B., Hohmann, S., Jensen, R. G., Bohnert, H. J. “Roles of sugar alcohols in osmotic stress adaptation: Replacement of glycerol by mannitol and sorbitol in yeast.” Plant Physiol. 1999; 121:45-52.

Wallach, M. “The importance of transmission-blocking immunity in the control of infections by apicomplexan parasites.” Int. J. Parasitol. 1997; 1159-1167.

Wang, C. C. “Validating targets for antiparasitic chemotherapy.” Parasitol. 1997; 114:S31-S44.

What is claimed is:

1. A method for identifying a compound having antiparasitic activity comprising:
a) exposing to the compound a parasite target gene-complemented microorganism growing in a selection conditions that inhibits the viability of non-complemented microorganisms;
b) comparing the viability of the microorganism in the selection conditions after exposure to the compound to the viability of the microorganism in the selection conditions lacking the compound to identify a compound that decreases viability of the microorganism, thereby identifying a compound having antiparasitic activity.

2. The method according to claim 1 further comprising comparing viability of the target gene-complemented microorganism after exposure to the compound in the absence of the selection conditions to viability of the microorganism in the absence of both the selection conditions and the compound.

3. The method according to claim 1 wherein the target gene expresses an enzyme in the mannitol pathway of a parasite.

4. The method according to claim 3 wherein the target gene is a mannitol-1-phosphate dehydrogenase (m1pdh) gene.

5. The method according to claim 3 wherein the parasite is in the phylum Apicomplexa.

6. The method according to claim 5 wherein the parasite is Eimeria tenella.

7. The method according to claim 1 wherein a non-complemented microorganism is unable to produce glycerol in response to osmotic stress.

8. The method according to claim 7 wherein the non-complemented microorganism is Saccharomyces cerivisiae mutated in the gene encoding glycerol-3-phosphate dehydrogenase.

9. The method according to claim 1 wherein the selection conditions comprise osmotic stress.

10. The method according to claim 9 wherein the selection conditions contain 1-2 M sodium chloride.

11. The method according to claim 9 wherein the selection conditions contain 1.5 M sodium chloride.

12. A method for identifying a compound useful as an antiparasitic drug comprising:
a) determining whether the compound decreases viability of a target gene-complemented microorganism by comparing the viability of a target gene-complemented microorganism growing in a selection medium after exposure to a compound to the viability of the target gene-complemented microorganism growing in the selection medium in the absence of the compound;
b) comparing viability of the target gene-complemented microorganism after exposure to the compound in the absence of the selection conditions to viability of the microoganism in the absence of both the selection medium and the compound, thereby identifying a compound useful as an antiparasitic drug.

13. The method according to claim 12 wherein the target gene expresses an enzyme of the mannitol pathway of a parasite.

14. The method according to claim 13 wherein the target gene is a mannitol-1-phosphate dehydrogenase (m1pdh) gene.

15. The method according to claim 13 wherein the parasite is in the phylum Apicomplexa.

16. The method according to claim 15 wherein the parasite is Eimeria tenella.

17. The method according to claim 12 wherein the non-complemented microorganism is unable to produce glycerol in response to osmotic stress.

18. The method according to claim 17 wherein the non-complemented microorganism is Saccharomyces cerivisiae mutated in the gene encoding glycerol-3-phosphate dehydrogenase.

19. The method according to claim 12 wherein the selection medium comprise osmotic stress.

20. The method according to claim 19 wherein the selection medium contains 1-2 M sodium chloride.

21. The method according to claim 19 wherein the selection medium contains 1.5 M sodium chloride.

22. A method of screening for a compound or identifying a compound that inhibits an essential parasite gene product required for parasite viability, said method comprising:
(a) rendering a microorganism incapable of growing under test conditions;
(b) producing a target gene-complemented microorganism by complementing the microorganism of step (a) with parasite gene encoding an parasite gene product that enables the microbial strain to grow in test conditions;
(c) exposing the target gene-complemented microorganism under test conditions to a compound to be tested for parasite gene product inhibitory properties and comparing the viability of the target gene-complemented microorganism exposed to the compound to the viability of the target gene-complemented microorganism in the absence of the compound.
(d) determining whether the compound inhibits growth of the target gene-complemented microorganism, thereby identifying a compound that inhibits an essential parasite gene product.

23. The method according to claim 22 wherein the test conditions comprise selection conditions containing 1.5 M sodium chloride.

24. The method according to claim 23 further comprising comparing viability of the target gene-complemented microorganism after exposure to the compound in the absence of the selection conditions to viability of the target gene-complemented microorganism in the absence of both the selection conditions and the compound.

25. The method according to claim 22 wherein the essential parasite gene expresses an enzyme of the anabolic mannitol pathway of a parasite.

26. The method according to claim 25 wherein the parasite gene is a mannitol 1-phosphate dehydrogenase (m1pdh) gene.

27. The method according to claim 25 wherein the parasite is in the phylum Apicomplexa.

28. The method according to claim 27 wherein the parasite is Eimeria tenella.

29. The method according to claim 22 wherein the microorganism is unable to produce glycerol in response to osmotic stress.

30. The method according to claim 29 wherein the microorganism is Saccharomyces cerivisiae mutated in the gene encoding glycerol-3-phosphate dehydrogenase.

31. A polypeptide sequence comprising SEQ ID NO: 6.

32. A polynucleotide sequence encoding, due to the degeneracy of the genetic code, the polypeptide sequence of claim 31.

33. The polynucleotide sequence of claim 32 wherein the sequence is SEQ ID NO: 5.

34. An expression vector comprising the polynucleotide sequence of claim 32.

35. A gene-complemented microorganism which has been transformed with the polynucleotide sequence of claim 32.

37. The microoganism of claim 35 wherein the micoroganism is a yeast.

38. The microorganism of claim 37 wherein the yeast is S. cerevisiae which, without the gene-complementation, is deficient at managing osmotic stress.

39. The microorganism of claim 38 wherein the uncomplemented microorganism is Δgdp1.

40. The microorganism of claim 39 wherein the microorganism is Δgdp1-pYES2/m1pdh.


SEQUENCE LISTING (8 sequences)

SEQ ID NO: 1 - DNA, 20 bp, Artificial Sequence Primer
  misc_feature: Primer used for deletion of GPD1 from yeast and complementary to 3′ region.
agaagttagtacaggccgtc

SEQ ID NO: 2 - DNA, 31 bp, Artificial Sequence Primer
  misc_feature: Primer used for deletion of GPD1 from yeast and complementary to 5′ region.
gaagatcttcaatatttgtgtttgtggaggg

SEQ ID NO: 3 - DNA, 30 bp, Artificial Sequence Primer
  misc_feature: Sense primer used for cloning of E. tenella M1PDH.
ctctgtcttggcaccatggctgctcctggc

SEQ ID NO: 4 - DNA, 30 bp, Artificial Sequence Primer
  misc_feature: Antisense primer used for cloning of E. tenella M1PDH.
tgctgcagcagcctgtatgcagccccaagt

SEQ ID NO: 5 - DNA, 1857 bp, Eimeria tenella
  CDS (1)..(1857): Cloned and isolated from sporulated oocyst by Nulf et al. (1999), Pharmacia & Upjohn Animal Health, unpublished.
atggctgctcctggctgcgaagctcctgctgcagaccacgaggcgctgcacctcgcccgccccgttttcgaagtcctcgaccaccccgccattctccgctttcgaaaagactaccagctgcaccggctgggctacgcgaagggggcgaagggggaagtgggtgcagcgctgcgcaaagtcgcggagaagcagcagcagcagcagcagggaggtgtggggccccacacttctttgctgccagaaggcatttcgggaaatgaagcagcaaacttgctttctccttttgctgcttatgcagcagattctccctttgccacagatgaggcccttttggctcagatcgccaggtggcgaatggcttaccagaacttcaggacgggagctgcacgcggagccgcaggtgctctggaagagatatcaaaaggcctgagcctgaaggcgctgaagctgctgccggtggtgcagctagctgaagtgctgcagcgcgacaagcagccaaaagggccccacaagttgctgccaactccccacctgcacttcggggccggcaagttgggctgcggccttgtgctgcaggccatgctgcgcagcaacgtcgaagacctcgtcgtgctgcagcgccagtcagaggacttcgagccgctgctttctgctgctaagcgcgaaggcattcctgtgtttgtaaatggcgaaaaggtttgtgagcttcgggtgtacgtacaccccaacgagatagaggaactcattcaaaagctcgaaaataaaaagctccagaattccaaatctccagggcatacaagcactcacctggtattgacaaacgacagcgaatttgccctcaaacttgcaaaagctgcaaagagcctcagctgctctttaggcccagctatggagaccgcgctgctgccgcttctctcccagctacccgacacgccgaagaccgaacaggtgctgctctacgcctgcgaaaacgatcatgctgctgttgcgcgtctgaaggagcagctgcagcaaaaggttttggtgctgccctgcatggttgataggatttgtgctgctaggaatatctttgctgataagattgaagtctcagcagaaccttatgaaggagaaattgtggttttggagaggccccagtttgcgcctccgccgccctttgggggccccaatgtgcatgcgccccagcttcgggcggcggcgcactacctctgccagcgaaagattggacttgttaatggcatgcatacaactctagcttttttaactgcatgcagcagacgcgacgagctgcttgctgctgctaaggaagcagcaaagccaaggacggcccgcagcagcagcagcagcagcagcagcggcagcagcagcagcgcagacgccgagggcagcagcagcagcagcagcagcagcgagagtcccgacccgctgctggtggcgctgcgctctgtcccgctgctgaaagagacagaaatgcagcagcagcaaaaagaaacaatgtgggcctggctcactgccaggtgtctccaagttctttgggagcacgacaaagaagtcatcaagaggacacacaatctgaagacagacgacgaggtggtcgccatgctgcttaactacggaaagaagactctagcccgattctccaccgtcgatgacactgcgggcagagttttgggcgggggagttgtaaacaggttccatacgcgtctgttgactatttatacatttttggagcagcacatgtttgggtctgtgccactggcatcgactttgctcaaacacgctaccatcagcgccttcgagatgatagacgaagtgcggcggcttgttgaccagtccagaatatttgtagagaaataa

SEQ ID NO: 6 - protein, 618 aa, Eimeria tenella
MAAPGCEAPAADHEALHLARPVFEVLDHPAILRFRKDYQLHRLGYAKGAKGEVGAALRKVAEKQQQQQQGGVGPHTSLLPEGISGNEAANLLSPFAAYAADSPFATDEALLAQIARWRMAYQNFRTGAARGAAGALEEISKGLSLKALKLLPVVQLAEVLQRDKQPKGPHKLLPTPHLHFGAGKLGCGLVLQAMLRSNVEDLVVLQRQSEDFEPLLSAAKREGIPVFVNGEKVCELRVYVHPNEIEELIQKLENKKLQNSKSPGHTSTHLVLTNDSEFALKLAKAAKSLSCSLGPAMETALLPLLSQLPDTPKTEQVLLYACENDHAAVARLKEQLQQKVLVLPCMVDRICAARNIFADKIEVSAEPYEGEIVVLERPQFAPPPPFGGPNVHAPQLRAAAHYLCQRKIGLVNGMHTTLAFLTACSRRDELLAAAKEAAKPRTARSSSSSSSSGSSSSADAEGSSSSSSSSESPDPLLVALRSVPLLKETEMQQQQKETMWAWLTARCLQVLWEHDKEVIKRTHNLKTDDEVVAMLLNYGKKTLARFSTVDDTAGRVLGGGVVNRFHTRLLTIYTFLEQHMFGSVPLASTLLKHATISAFEMIDEVRRLVDQSRIFVEK

SEQ ID NO: 7 - DNA, 2709 bp, Eimeria tenella
  CDS (339)..(2192)
gaaagaatcttttcgtattccaaacttgaaattaattttgttatttccttgcaaacctcgagcttgtcttcgcctgcagtttccgcgcatttcctgtttttcagcagaagcctctgccaagtgtgcaccgagagctgaaacggctgcggatcgccgcagttcatgttttccgtgactgctttcgttcaagttctgcatttgccgcgtaggccgctaaaaagccaccaacctggagctgctgaaaaggcttttctttggaattgtattcgcctgcaattgggcctttggctgcagcaatagggccgcgtgttccctcgctttccctctgtcttggcaccatggctgctcctggctgcgaagctcctgctgcagaccacgaggcgctgcacctcgcccgccccgtttttgaagtcctcgaccaccccgccattctccgctttcgaaaagactaccagctgcaccggctgggctacgcgaagggggcgaagggggaagtgggtgcagcgctgcgcaaagtcgcggagaagcagcagcagcagcagcagggaggtgtggggccccacacttctttgctgccagaaggcatttcgggaaatgaagcagcaaacttgctttctccttttgctgcttatgcagcagattctccctttgccacagatgaggcccttttggctcagatcgccaggtggcgaatggcttaccagaacttcaggacgggagctgcacgcggagccgcaggtgctctggaagagatatcaaaaggcctgagcctgaaggcgctgaagctgctgccggtggtgcagctagctgaagtgctgcagcgcgacaagcagccaaaagggccccacaagttgctgccaactccccacctgcacttcggggccggcaagttgggctgcggccttgtgctgcaggccatgctgcgcagcaacgtcgaagacctcgtcgtgctgcagcgccagtcagaggacttcgagccgctgctttctgctgctaagcgcgaaggcattcctgtgtttgtaaatggcgaaaaggtttgtgagcttctggtgtacgtacaccccaacgagatagaggaactcattcaaaagctcgaaaataaaaagctccagaattccaaatctccagggcatacaagcactcacctggtattgacaaacgacagcgaacttgccctcaaacttgcaaaagctgcaaagagcctcagctgctctttaggcccagctatggagaccgcgctgctgccgcttctctcccagctacccgacacgccgaagaccgaacaggtgctgctctacgcctgcgaaaacgatcatgctgctgttgcgcgtctgaaggagcagctgcagcaaaaggttttggtgctgccctgcatggttgataggatttgtgctgctaggaatatctttgctgataagattgaagtctcagcagaaccttatgaaggagaaattgtggttttggagaggccccagtttgcgcctccgccgccctttgggggccccaatgtgcatgcgccccagcttcgggcggcggcgcactacctctgccagcgaaagattggacttgttaatggcatgcatacaactctagcttttttaactgcatgcagcagacgcgacgagctgcttgctgctgctaaggaagcagcaaagccaaggacggcccgcagcagcagcagcagcagcagcagcggcagcagcagcagcgcagacgccgagggcagcagcagcagcagcagcagcgagagtcccgacccgctgctggtggcgctgcgctctgtcccgctgctgaaagagacagaaatgcagcagcagcaaaaagaaacaatgtgggcctggctcactgccaggtgtttccaagttctttgggagcacgacaaagaagtcatcaagaggacacacaatctgaagacagacgacgaggtggtcgccatgctgcttaactacggaaagaagactctagcccgattctccaccgtcgatgacactgcgggcagagttttgggcgggggagttgtaaacaggttccatacgcgtctgttgactatttatacatttttggagcagcacatgtttgggtctgtgccactggcatcgactttgctcaaacacgctaccatcagcgccttcgagatgatagacgaagtgcggcggcttgttgaccagtccagaatatttgtagagaaataaacttggggctgcatacaggctgctgcagcagctgcgactttgcagctgctgcagcagctgcaaagtcagcagcaactgtagcagcaactgcagcagcagctgcaaagacagcagcgactgcagcagcagctgcaaagacagcagcgactgcagcagcagctgcaaagacagcagcaactgcagcagcagctgcaaaagcagcagcaactgcagcagcggctaaaggaacaacttcggcgtcagtggctgcagcaggtacagctgaagcaacagcaaaaccagcagctgcgcagcagttagcagcgtcagcagaagctgctacagcacttgctgaagcagcagcagcagcacctgctgcagcaacaagttgaatttagtggcgcctacgtagcagcagctgctaatgcatcatcaactgcagaagcaacagctgttaacgtgtttgtatagagacccaaatgctgctgcaacattgttgaacttggttaagcctccgctggctgcagcaaaaaaaaaa

SEQ ID NO: 8 - protein, 617 aa, Eimeria tenella
MAAPGCEAPAADHEALHLARPVFEVLDHPAILRFRKDYQLHRLGYAKGAKGEVGAALRKVAEKQQQQQQGGVGPHTSLLPEGISGNEAANLLSPFAAYAADSPFATDEALLAQIARWRMAYQNFRTGAARGAAGALEEISKGLSLKALKLLPVVQLAEVLQRDKQPKGPHKLLPTPHLHFGAGKLGCGLVLQAMLRSNVEDLVVLQRQSEDFEPLLSAAKREGIPVFVNGEKVCELLVYVHPNEIEELIQKLENKKLQNSKSPGHTSTHLVLTNDSELALKLAKAAKSLSCSLGPAMETALLPLLSQLPDTPKTEQVLLYACENDHAAVARLKEQLQQKVLVLPCMVDRICAARNIFADKIEVSAEPYEGEIVVLERPQFAPPPPFGGPNVHAPQLRAAAHYLCQRKIGLVNGMHTTLAFLTACSRRDELLAAAKEAAKPRTARSSSSSSSSGSSSSADAEGSSSSSSSESPDPLLVALRSVPLLKETEMQQQQKETMWAWLTARCFQVLWEHDKEVIKRTHNLKTDDEVVAMLLNYGKKTLARFSTVDDTAGRVLGGGVVNRFHTRLLTIYTFLEQHMFGSVPLASTLLKHATISAFEMIDEVRRLVDQSRIFVEK